(19) United States
(12) Plant Patent Application Publication
(10) Pub. No.: US 2009/0031461 P1
(43) Pub. Date: Jan. 29, 2009

(54) 'Primadonna' southern highbush blueberry

(76) Inventor: Paul M. Lyrene, Gainesville, FL (US)

Correspondence Address: JONDLE & ASSOCIATES P.C., 858 HAPPY CANYON ROAD SUITE 230, CASTLE ROCK, CO, 80108, US

(21) Appl. No.: 11/288,765

(22) Filed: Nov. 30, 2005

(51) Int. Cl. A01H 5/00

(52) U.S. Cl. PLT/157

(57) ABSTRACT

A new and distinct low-chill southern highbush (Vaccinium corymbosum) cultivar. Its novelty lies in the following unique combination of features: 1. Has a low chilling requirement. 2. Has a vigorous bush, between upright and spreading. 3. Produces large berries with excellent scar and firmness. 4. Ripens the first 25% of its berries by April 12 in north Florida in an average year.

### CROSS-REFERENCE TO RELATED APPLICATIONS

None.

### STATEMENTS REGARDING FEDERALLY SPONSORED RESEARCH

None.

### LATIN NAME OF THE GENUS AND SPECIES

Vaccinium corymbosum L.

### VARIETY DENOMINATION

‘Primadonna’.

### BACKGROUND OF THE INVENTION

The invention relates to a new and distinct variety of southern highbush blueberry (Vaccinium corymbosum L) hybrid named ‘Primadonna.’ ‘Primadonna’ is a blueberry clone that is distinguished by its low chilling requirement and by its ability to produce large, firm berries during April and early May when grown in north Florida. Several thousand plants of ‘Primadonna’ have been propagated by softwood cuttings at Gainesville, Fla., and the resulting plants have all been phenotypically indistinguishable from the original plant. Contrast is made to ‘Star’ (U.S. Plant Pat. No. 10,675), an important variety widely planted in Florida and Georgia for early-season blueberry production. The new variety is important because it ripens earlier than Star and is more resistant to cane canker (Botryosphaeria dothidia).

### BRIEF SUMMARY OF THE INVENTION

‘Primadonna’, when grown in north Florida, is distinguished from all other blueberry plants by the following combination of characteristics: has good resistance to cane canker (Botryosphaeria corticis); ripens 9 to 14 days earlier than ‘Star’; produces large berries with excellent picking scar and firmness; produces berries in loose clusters which, when mature, detach easily from the stems.

### ORIGIN OF THE VARIETY

‘Primadonna’ originated as a seedling from the cross O'Neal (unpatented)×FL87-286 (unpatented) made as part of the University of Florida breeding program in a greenhouse in March, 1988. The seedling was first fruited in a high-density field nursery in the spring of 1990. The plant was propagated by softwood cuttings in June, 1992, and a 10-plant plot was established at the University of Florida Horticultural Unit in Gainesville, Florida in January 1994. Based on the high fruit quality and early ripening, the plant was propagated by cuttings and planted at 5 field sites from central Florida to southeast Georgia between 1997 and 2000. A plot of 200 plants established in a commercial field in January, 1999 at Windsor, Florida has been carefully observed through 6 fruiting cycles.

### BRIEF DESCRIPTION OF THE DRAWING

The color designations in this specification are from “The Pantone Book of Color” ( by Leatrice Eiseman and Lawrence Herbert; Harry N. Abrams, Inc., Publishers, New York, 1990). Where the Pantone color designations differ from the colors in the Drawings, the Pantone colors are the more accurate.

FIG. 1 shows flower clusters of ‘Primadonna’ during February on a mature plant growing in the field. Cold weather during the flowering period imparts a slight pinkish tinge to the corollas of unopened flowers.

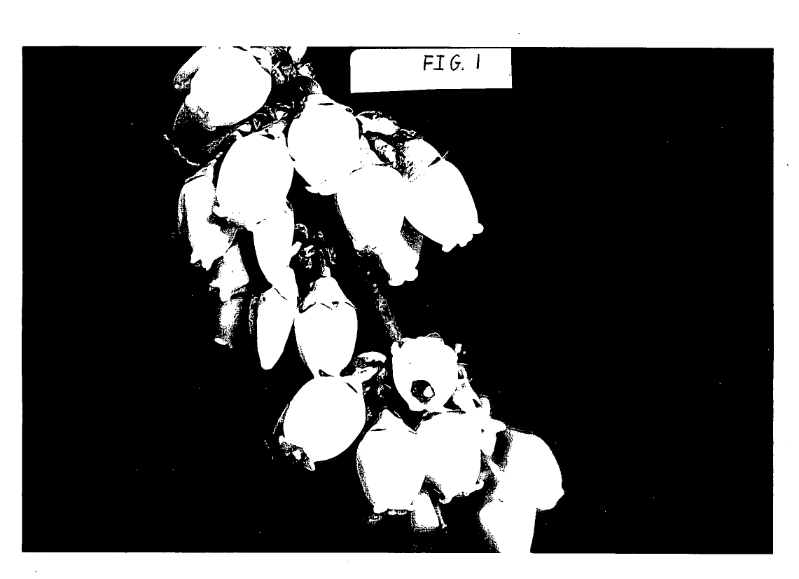

FIG. 2 shows a row of six-year-old plants at the time of flowering in February. The plants are about 2 m tall.

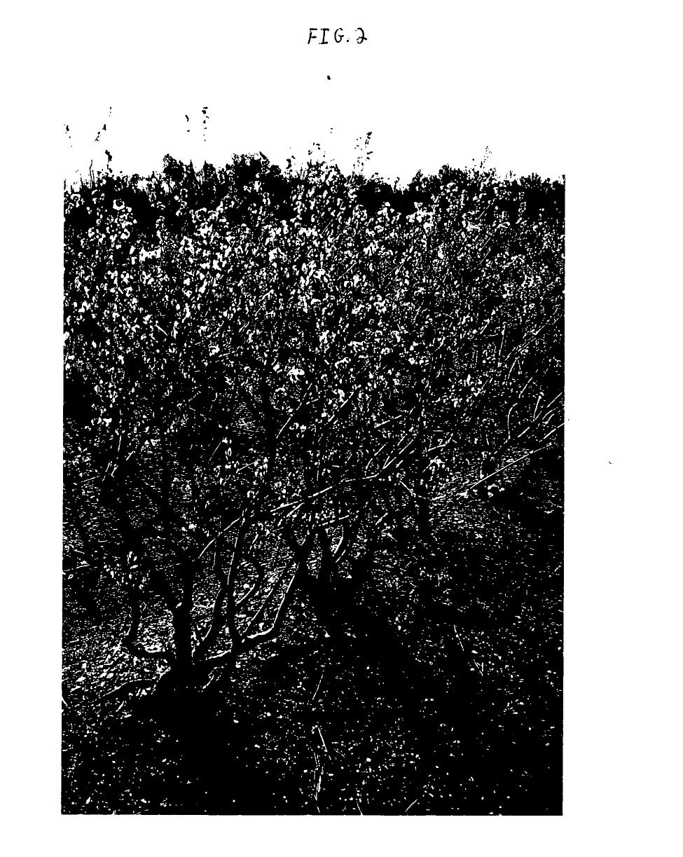

FIG. 3 shows at close range a cluster of berries that is beginning to ripen in the field. Slight frost damage is visible at the calyx end of several berries.

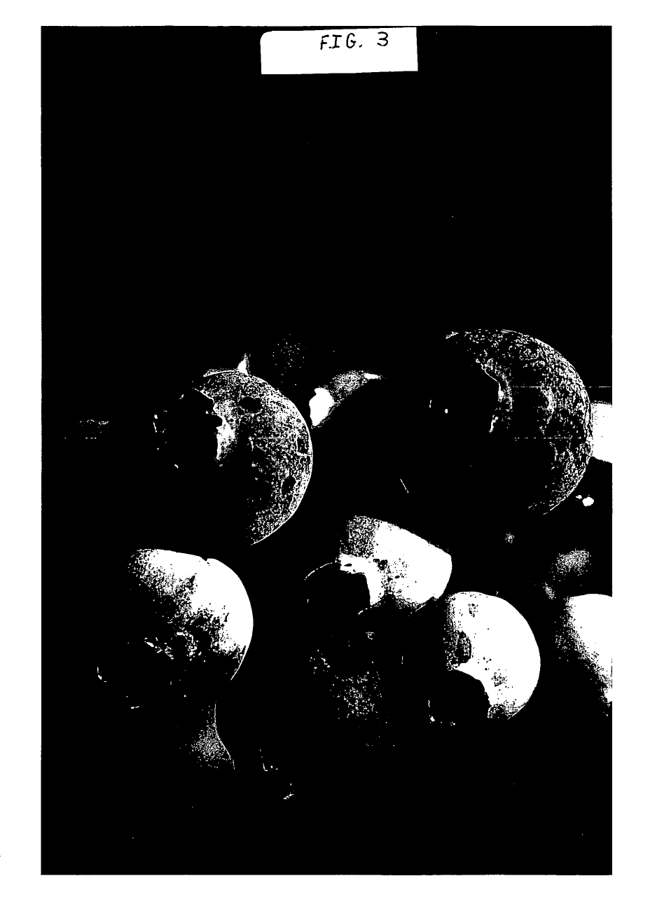

FIG. 4 shows, at close range, the mature berries. The small picking scar and the rather undeveloped calyx lobes can be seen.

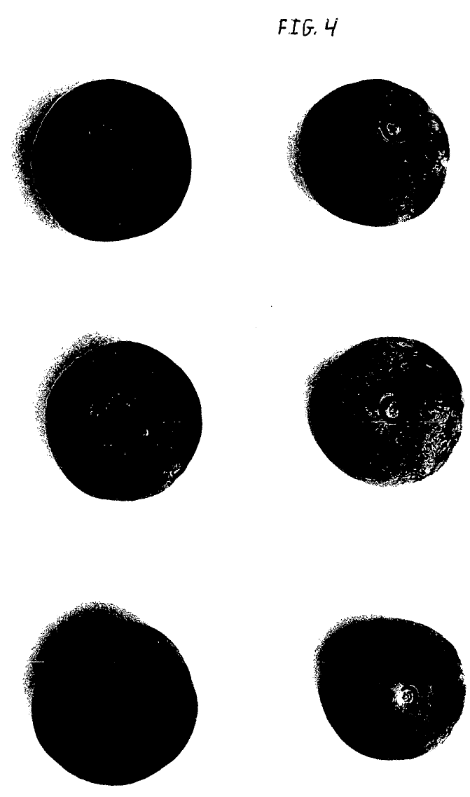

### DETAILED BOTANICAL DESCRIPTION

Market class: ‘Primadonna’ produces southern highbush blueberries suitable for both the fresh and processed fruit markets.
Bush: The following descriptions are based on a plot of 200 six-year-old ‘Primadonna’ plants growing in a test plot in a commercial blueberry planting near Windsor in north Florida.
   Plant height.—2 m.
   Canopy diameter.—1.7 m.
   Plant vigor.—High. Somewhat more vigorous than ‘Star’.
   Growth habit.—Between upright and spreading.
   Flower bud density (number) along flower twigs in January.—Medium. Produces more flower buds on upright shoots than ‘Star’.
   Twigginess.—Medium to low.
Trunk and branches:
   Suckering tendency.—Plants have an average of 5 to 7 major canes per plant from a base 30 cm in diameter on 6-year-old plants.
   Surface texture of new wood (9 months old or less) observed in March.—smooth.
   Surface texture of strong, 1-year-old shoots observed in March.—rough but becoming smooth by exfoliation of rough bark in vertical strips.
   Surface texture of 3-year-old and older wood surface.—Rough to smooth; continually exfoliating vertical strips of rough bark to leave a smooth surface.
   Color of 1-month-old new shoots observed March 30.—“Green Moss”, Pantone 17-0636.
   Color of previous-summer smooth twigs observed March 30.—“Pampas”, Pantone 14-0826.
   Color of 1-year-old rough bark.—“Slate Green”, Pantone 16-0713.
   Color of 3-year-old rough-textured canes.—“Cement”, Pantone 14-0708.
   Internode length on strong, new shoots measured March 30.—Mean=1.9 cm.
   Relative time of leafing vs flowering.—Tends to flower before it begins to leaf when coming out of dormancy. Commercially in north and central Florida, plants are often sprayed with formulations of hydrogen cyanomide in midwinter to enhance early leafing.
Flowers:
   Fragrance.—Faint smell of Camellia flowers.
   Pedicel length at time of anthesis.—Median=4 mm.
   Peduncle length at time of anthesis.—Variable; median=11 mm.
   Flower shape.—Urceolate.
   Pollen staining with 2% acetocarmine.—This is a measure of potential pollen fertility. 99%.
   Abundance of pollen shed.—High.
   Color of dried pollen.—‘Yolk yellow’, Pantone 14-0846.
   Flower length, pedicel attachment point to corolla tip excluding the pedicel.—12 mm.
   Length of corolla tube.—8 to 9 mm.
   Diameter of corolla tube at widest point.—6 mm.
   Style length.—top of ovary to stigma tip. 9 mm.
   Calyx diameter at anthesis.—tip of lobe to tip of opposite lobe. 5 mm.
   Corolla aperture diameter.—3 mm.
   Calyx surface.—Smooth.
   Corolla color at anthesis.—‘Turtledove’, Pantone 12-5202.
   Corolla surface texture.—Smooth.
   Calyx color at anthesis.—‘Cedar’, Pantone 16-0526.
   Pistil color at anthesis.—‘Golden green’, Pantone 15-0636.
   Pedicel and peduncle color.—‘Willow green’, Pantone 15-0525.
   Flowering period.—mean date of 50% open flower at Windsor, Fla. Feb. 16 compared to Feb. 25 for ‘Star’.
   Flower cluster (tight, medium, open).—Medium.
   Number of flowers per cluster.—Median=7.
   Location of tip of stigma relative to lip of the corolla.—Stigma tip lacks 1 mm of reaching the end of the corolla tube.
Berry:
   Mean date of first commercial harvest (25% of fruit ripe) at Windsor, Fla.—April 12.
   Mean date of last commercial harvest.—May 1.
   Diameter of calyx aperture on mature berry.—6 mm.
   Calyx lobes on mature berry.—size and shape. Small, irregular, not prominent.
   Berry cluster.—(tight, medium, or loose). Loose.
   Pedicel length on ripe berry.—Variable; median length 5 to 6 mm.
   Peduncle length at the time berries are ripe.—Variable; median length 10 mm.
   Number of ripe berries per cluster.—Median 6 to 7.
   Mean berry weight on well-pruned plants.—Mean 2.13 g.
   Mean berry height.—15 mm.
   Mean berry width.—17 mm.
   Surface color of ripe berry while on plant.—“Gull”, Pantone 17-3802.
   Surface color of ripe berry after harvest and packing.—“Steel Gray”, Pantone 18-4005.
   Surface color of ripe berry after polishing.—“Shale”, Pantone 19-3903.
   Internal flesh color of ripe berry.—“White Jade”, Pantone 12-0315.
   Berry surface wax.—how much and how persistent. Medium amount, medium persistence during handling.
   Berry pedicel scar.—Small and dry.
   Berry firmness.—High.
   Berry flavor.—Sweet, low in acidity.
   Berry texture.—Small seeds, tender skin, not gritty.
   Color of dried seeds.—‘Aztec’, Pantone 18-1130.
   Weight of well-developed dried seeds.—0.47 mg.
   Length of well-developed, dried seeds.—1.8 mm.
   Width of well-developed, dried seeds.—1.1 mm.
Physiological characteristics:
   Chilling requirement.—Flower buds break dormancy well after 300 hrs. below 7° C. Spring leafing is best if the plant receives at least 400 hrs. below 7° C.
   Cold hardiness.—Flowers and fruit hardy to −3° C. The plant, during winter dormancy is hardy to −15° C.
   Regrowth after May hedging.—Blueberries in north Florida are normally top-pruned mechanically (hedged) shortly after harvest. ‘Primadonna’ produces an unusually prolific regrowth of new shoots from the top of the bush after May hedging.
   Productivity.—In northeast Florida, about 5 pounds of berries per bush on plants 4 years old and older.
   Ease of propagation.—‘Primadonna’ is easy to propagate from softwood cuttings. Several hundred plants have been propagated by this method in Gainesville, Fla., and all are typical of the variety.
Resistance to diseases, insects, mites:
   Phytophthora root rot.—High.
   Stem blight (Botryosphaeria dothidea).—Medium to high.
   Cane canker (Botryosphaeria corticis).—High.
   Fungal leaf spots.—Above average resistance for southern highbush blueberry. Fungicidal control of leaf diseases normally increases yields of southern highbush blueberries where summers are rainy and humid and pathogens are present.
   Overall survival in the field.—Medium. Plant survival in the field is about average for southern highbush blueberry varieties grown in Florida.

1. A new and distinct southern highbush blueberry plant, substantially as illustrated and described, characterized by a vigorous, low-chill bush that produces large berries with excellent scar and firmness during April in north Florida.
